(19) United States
(12) Patent Application Publication
(10) Pub. No.: US 2004/0069312 A1
(43) Pub. Date: Apr. 15, 2004

(54) Method of operating for anal fistula

(76) Inventor: Yoshihiro Ohmi, Ibaraki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/268,102

(22) Filed: Oct. 10, 2002

(51) Int. Cl. A61B 19/00

(52) U.S. Cl. 128/898

(57) ABSTRACT

The present invention comprises a constitution of a method of operating for an anal fistula wherein said anal fistula vanishes away by closing a primary opening of the purulent anal fistula developed round an anus, a constitution of a method of operating or an anal fistula wherein a primary opening is closed and the lower portion of the anal fistula is also cut away at the same time when secondary openings are developed at two portions of the anal fistula developed round an anus, a constitution of a method of operating for an anal fistula wherein a primary opening of the purulent anal fistula developed round an anus is closed and a part of a periproctal abscess is also incised at he same time, a constitution of a method of operating for an anal fistula wherein an internal hemorrhoid and an external hemorrhoid are ligated and cut away and a primary opening of the anal fistula developed in the anus portion is also closed at the same time when the anal fistula having the primary opening and a secondary opening in the anus portion and the internal hemorrhoid and the external hemorrhoid are developed, and a constitution of a method of operating for an anal fistula wherein a primary opening of one anal fistula is closed to be removed and a primary opening of the other anal fistula is also closed to be removed at the same time when the anal fistulas are developed at two portions round an anus.

### TECHNICAL FIELD

The present invention relates to a method of operating for an anal fistula developed at an anus portion.

### BACKGROUND ART

Heretofore, it has been considered that resection or release of a primary opening through which bacteria invade, an inflamed anal gland between internal and external sphincters, and surrounding tissue is necessary in a radical operation for an anal fistula, and therefore the operation has been performed employing incisional surgery such as resection, release.

However, there has been a problem that fecal incontinence, soiling, etc. May occur as a secondary disease even by means of the incisional surgery, which is a conventional method of operation for anal fistulas.

Although the method of operation has been improved to cope with liability to anal functional hazards in the case of women's frontal anal fistulas, all the discussions on the radical operations for anal fistulas have been focused until now on how to cut away fistulous tract portions between internal anal sphincters and external anal sphincters.

Accordingly, in order to solve the above problem, the present invention aims to provide a method of operation where an anal fistula can be mitigated by closing the primary opening, which is an inlet of bacteria if the cause of the anal fistula is infection of an anal gland.

### DISCLOSURE OF THE INVENTION

Aspects of the present invention are directed to a method of operating for an anal fistula, comprising the step of closing a primary opening of the purulent anal fistula developed round an anus to vanish away the anal fistula; a method of operating for an anal fistula, comprising the steps of closing a primary opening, and cutting away a lower portion of the anal fistula, in the case that secondary openings are developed at two anal fistula portions developed round an anus; a method of operating for an anal fistula, comprising the steps of closing a primary opening of the purulent anal fistula developed round an anus, and incising a part of a periproctal abscess round the anus; a method of operating tar an anal fistula, comprising the steps of ligating and cutting away an internal hemorrhoid and an external hemorrhoid, and closing a primary opening of the anal fistula developed in ah anus portion, in the case that the anal fistula having the primary opening and a secondary opening in the anus portion is developed, and the internal hemorrhoid ad the external hemorrhoid are developed; and a method of operating for an anal fistula, comprising the steps of closing and removing a primary opening of one anal fistula, and closing and removing a primary opening of the other anal fistula, in the case that the anal fistulas are developed at two portions round an anus.

### BRIEF DESCRIPTION OF THE DRAWINGS

FIG. 1 is a longitudinal cross section showing 3 main diseases of an anal fistula.

FIG. 2 is a flow diagram showing generation of an anal fistula and a method of operating therefor.

FIG, 3 is an enlarged view of an anal fistula portion to be operated.

FIG. 4 is an enlarged bottom view of the anal fistula portion to be operated.

FIG. 5 is an enlarged view showing a state where a primary opening is closed.

FIG. 6 is an enlarged bottom view showing a state where the primary opening is closed.

FIG. 7 is a flow diagram showing a flow of another method of operation.

FIG. 8 is an enlarged view of an anal fistula portion to be operated.

FIG. 9 is an enlarged bottom view of the anal fistula portion to be operated.

FIG. 10 is an enlarged view showing a state where the operation has been performed.

FIG. 11 is an enlarged bottom view showing a state where the operation has been performed.

FIG. 12 is an enlarged view of an anal fistula portion to be operated.

FIG. 13 is an enlarged bottom view of the anal fistula portion to be operated.

FIG. 14 is an enlarged view showing a state where a primary opening is closed.

FIG. 15 is an enlarged bottom view showing a state where the primary opening is closed.

FIG. 16 is an enlarged view showing the case where an anal fistula to be operated has secondary openings developed at two portions.

FIG. 17 is an enlarged bottom view showing the case where the anal fistula to be operated has the secondary openings developed at the two portions.

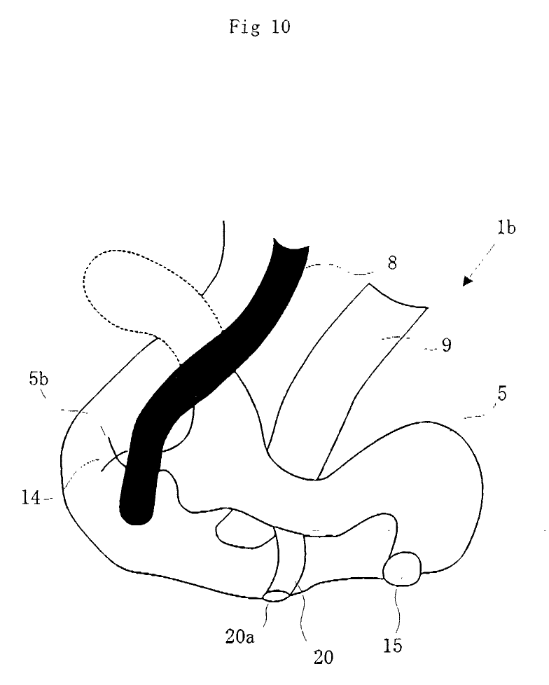

FIG. 10 is an enlarged view showing a slate where a primary opening is closed in the case where an anal fistula to be operated has secondary openings developed at two portions.

FIG. 19 is an enlarged bottom view showing a state where the primary opening is closed in the case where the anal fistula to be operated has the secondary openings developed at the two portions.

FIG. 20 are a bottom view of an anus at an acute period of an anal fistula, which is a periproctal abscess to be operated and a bottom view showing a state where a primary opening thereof is closed.

FIG. 21 are a bottom view of an anus to he operated where an internal hemorrhoid and an external hemorrhoid and an anal fistula are developed, resection of the internal hemorrhoid and an external hemorrhoid is carried out, and a primary opening of an anal fistula is closed.

FIG. 22 are a bottom view of an anus to be operated where an anal fistula and an anal fistula are developed and a bottom view showing a state where one anal fistula is incisionally released and a primary opening of the other anal fistula is closed.

FIG. 23 is a table showing the periods of relapse after closing the primary openings of the anal fistulas operated.

FIG. 24 is a table showing the frequencies of relapse depending on the types of the anal fistulas operated.

### BEST MODE FOR CARRYING OUT THE INVENTION

In the following, methods of operating for an anal fistula will be described in details on the basis of the appended drawings.

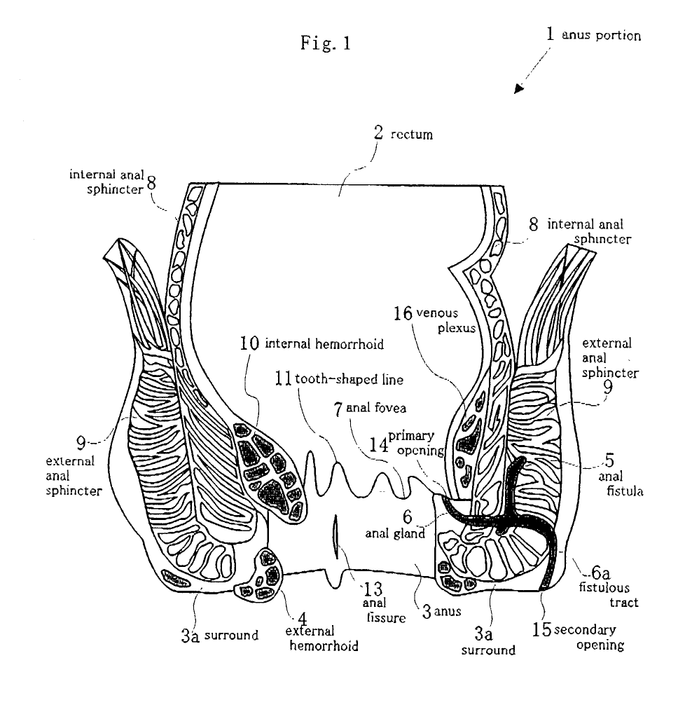

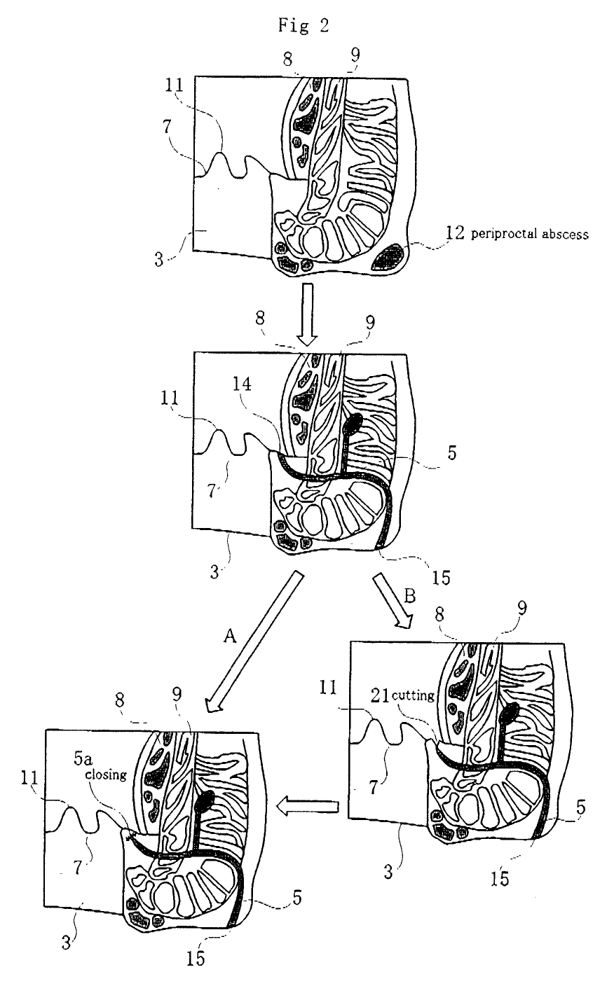

FIG. 1 is a longitudinal cross section showing 3 main piles. As shown in FIG. 1, 3 main piles refer to bleeding piles (anal fissure), hemorrhoid (blind piles) and an anal fistula (pus piles). As shown in FIG. 1, an anus portion 1 includes an anus 3 below a rectum 2, and an anal fovea 7 and a tooth-shaped line 11 between said rectum 2 and said anus 3.

Furthermore, there are an internal anal sphincter 8 outside both the rectum 2 and the anus 3, and an external anal sphincter 9 outside the internal anal sphincter 8. There are venous plexus 16, 16 on a level with the tooth-shaped line 11 inside the internal anal sphincter 8.

An anal fissure 13 (bleeding piles) is a piles which is formed due to tearing of an inner surface of the anus 3. Further, an external hemorrhoid 4 is a hemorrhoid, which is formed outside the anus 3. An internal hemorrhoid 10 is a hemorrhoid, which is formed protruding inward on an inner surface of the rectum 2. And, bacteria invade from the tooth-shaped line 11 into an anal gland 6 to be infected and to get purulent resulting in an abscess 12 or the anal fistula 5.

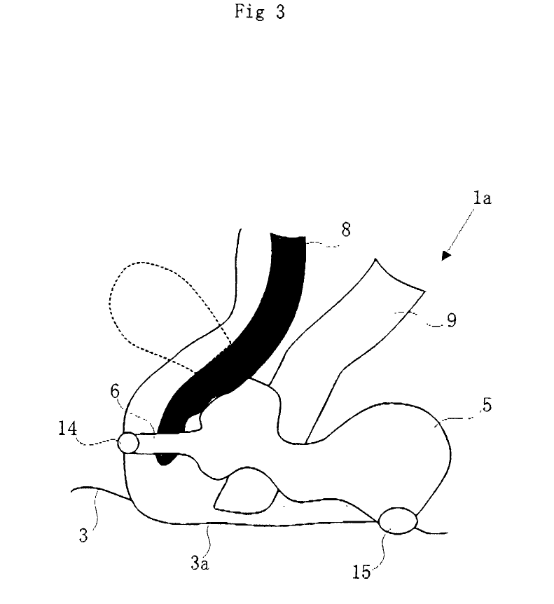

FIG. 2 is a flow diagram showing generation of an anal fistula and a method of operating therefor. Namely, it shows a flow of generation of a periproctal abscess 12→development into an anal fistula 5→an operation. The generation of the anal fistula 5 starts first from bacteria's invading into an anal fovea 7 to form a periproctal abscess 12 and the puss accumulated in the periproccal abscess 12 breaking a skin to communicate with the interior of an anus 3 and forming a fistulous tract 6a. A state where the fistulous tract 6a has got worse is called an anal fistula 5.

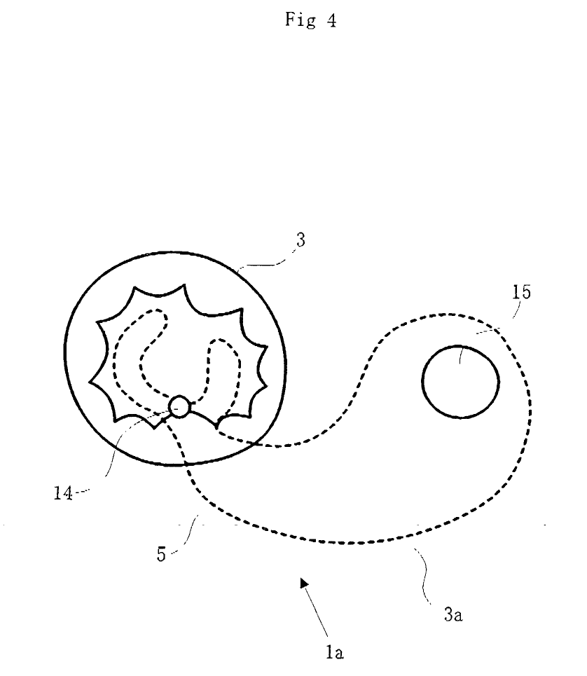

FIG. 3 is an enlarged view of an anal fistula portion to be operated, and FIG. 4 is an enlarged bottom view o f the anal fistula portion to be operated. As shown in FIGS. 3 and 4, an anal fistula having a primary opening 14 and a secondary opening 15 developed in a surround 3a of an anus 3 in an anal fistula portion 1a.

The anal fistula portion 1a is in a state where a periproctal abscess 12 is formed by bacteria which has entered from an anal fovea 7 and the pus accumulated in the periproctal abscess 12 breaks a skin to form a tract communicating with the interior of the anus 3 and the fistulous tract 6a gets worse to be the anal fistula 5.

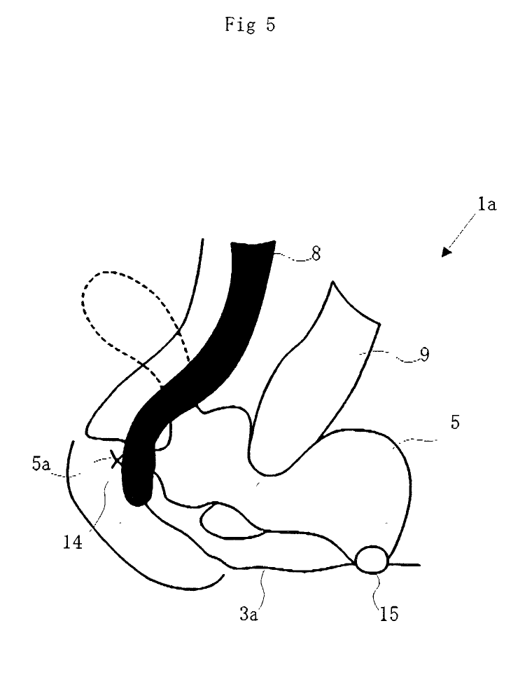

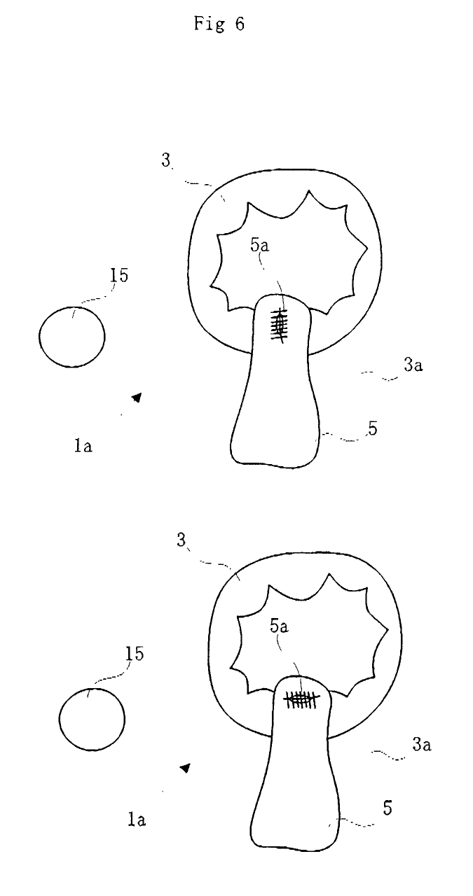

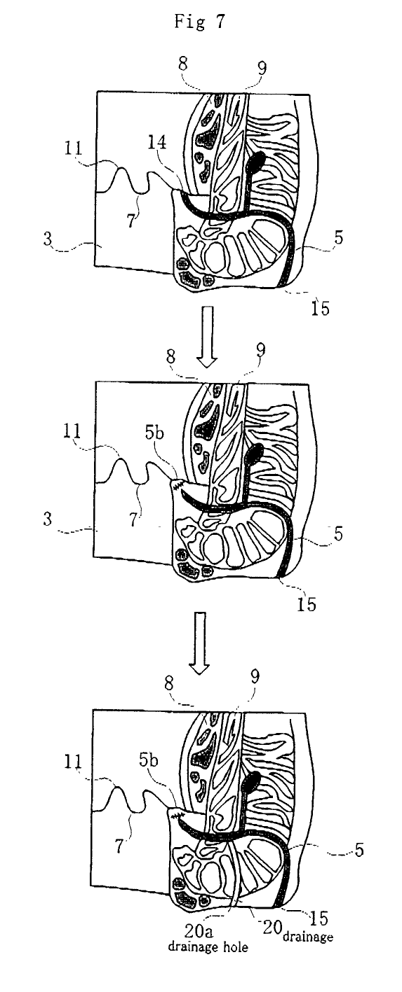

The anal fistula 5 refers to a state having got worse due to accumulation of pus in the fistulous tract 6a, and the primary open ng 14 and the secondary opening 15 are formed, and the primary opening 14 is formed on the anus 3 side and the secondary opening 15 is formed on the surround 3a side. Obviously, if the anal fistula 5 having formed fistulous tract 6a is left as it is, it may turn out a complicated branched state to develop into an anal fistula cancer, and therefore such an operation method as shown in FIGS. 5 and 6 is carried out.

FIG. 5 is an enlarged view showing a state where a primary opening of an anal fistula is closed, and FIG. 6 is an enlarged bottom view showing a state where a primary opening is closed. As shown in FIGS. 5 and 6, since the anal fistula 5 is an abscess which has go purulent by invasion of bacteria can be stopped getting worse by preventing the invasion of bacteria. Therefore, the invasion of bacteria is prevented by closing 5a the primary opening 14, which has been formed on the anus 3 side, and the anal fistula tends toward healing.

As shown in FIG. 6, a method of closing 5a includes a longitudinal suture and a lateral suture. Also as shown in FIG. 2, there are a method A where the primary opening 14 is closed 5a as it is and a method B where it is closed 5a after cutting it away once.

Another method of operating for an anal fistula will be described according to FIGS. 7 to 11. FIG. 7 is a flow diagram showing a flow of another method of operating for an anal fistula. An operation performed to promote early healing after closing 5b a primary opening 14 of the anal fistula 5.

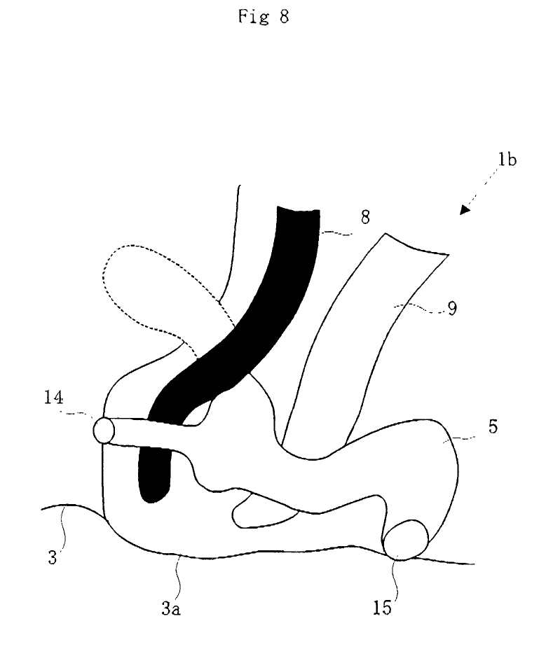

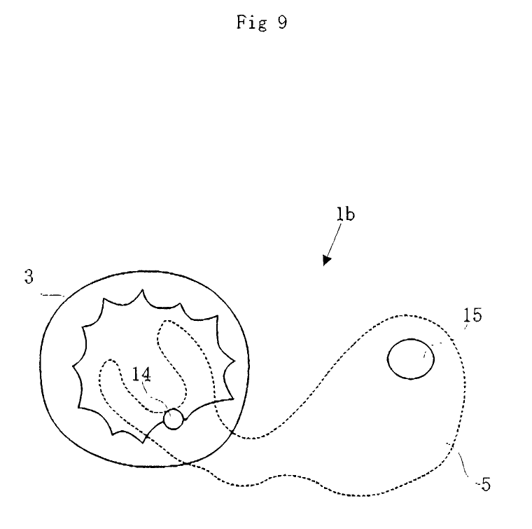

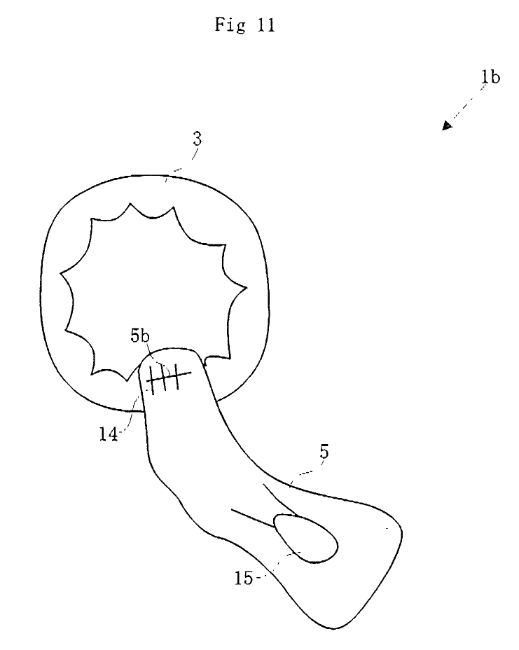

FIG. 8 is an enlarged view of an anal fistula portion to be operated, and FIG. 9 is an enlarged bottom view of the anal fistula portion, to be operated. As shown in FIGS. 8 and 9, an anal fistula 5 swelled with accumulation of pus is highly liable to turn out a branched arid complicated state to develop into an anal fistula cancer if it is left as it is without operation. Therefore, a method of operation shown in FIGS. 10 and 11 is performed.

FIG. 10 is an enlarged view showing a state where the operation has beer performed, and FIG. 11 is an enlarged bottom view showing a state where the operation has been performed. As shown in FIGS. 10 and 11, after a primary opening 14 is closed 5b to prevent invasion of bacteria, a drainage 20 and a drainage hole 20a are formed directly in an anal fistula 5 for the pus standing in the anal fistula 5 to be expelled easily out of the body.

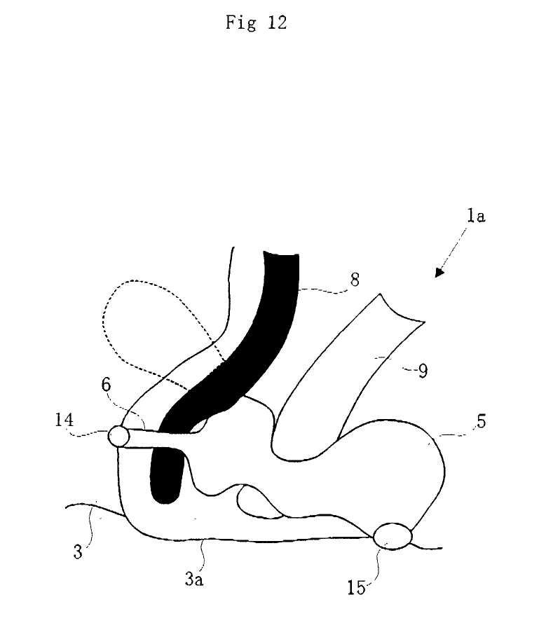

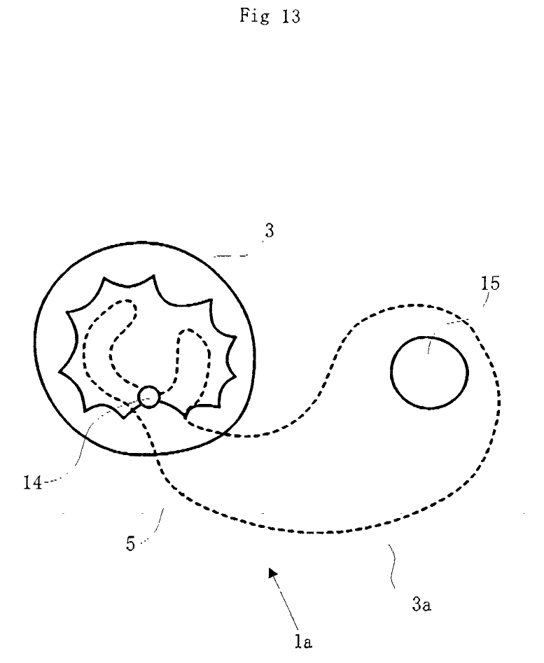

FIG. 12 is an enlarged view of an anal fistula portion to be operated, and FIG. 13 is an enlarged bottom view of the anal fistula portion to be operated. As shown in FIGS. 12 and 13, an anal fistula 5 having a primary opening 14 and a secondary opening 15 is developed in a surround 3a of an anus 3.

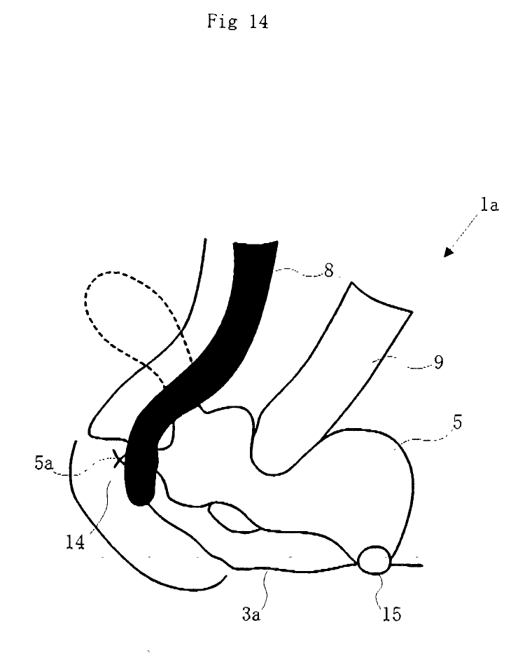

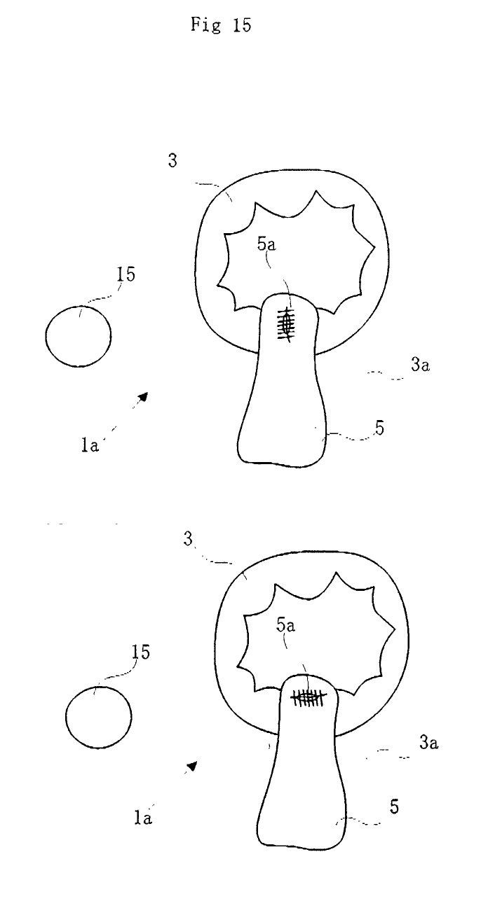

As shown in FIGS. 12 and 13, in an anal fistula portion 1a, the primary opening 14 and the secondary opening 15 of an anal gland 6 exist in the purulent anal fistula 5 infected with bacteria which has invade into the anal gland 6 through a tooth-shaped line 11 An the surround 3a of the anus 3. For that kind of anal fistula performed is such a method of operation as shown in FIGS. 14 and 15.

FIG. 14 is an enlarged view stowing a state where a prima nary opening is closed, and FIG. 15 is an enlarged bottom view showing a state where the primary opening is closed. As shown in FIGS. 14 and 15, by closing 5a the primary opening 11 of a purulent anal fistula 5 developed in a surround 3a of an anus 3, the purulent anal fistula 5 in the surround 3a of the anus 3 is vanished. 1y closing 5a the primary opening 14 of the anal fistula, the anal fistula 5 is healed. Further, closing 5a includes a longitudinal suture and a lateral suture.

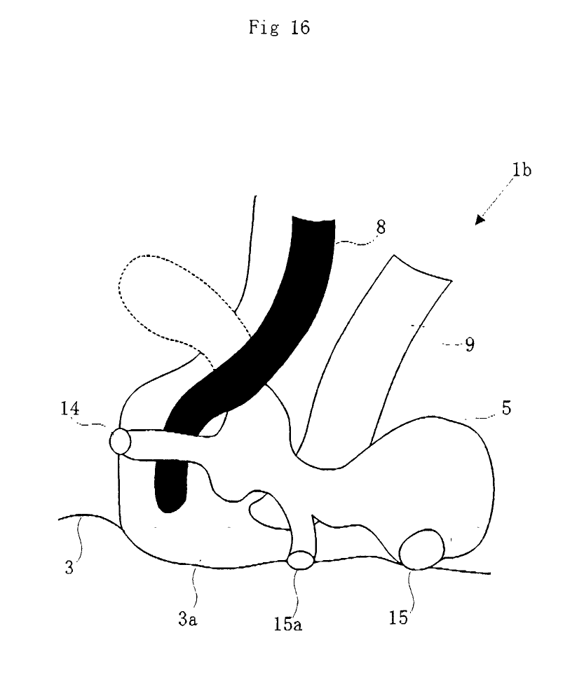

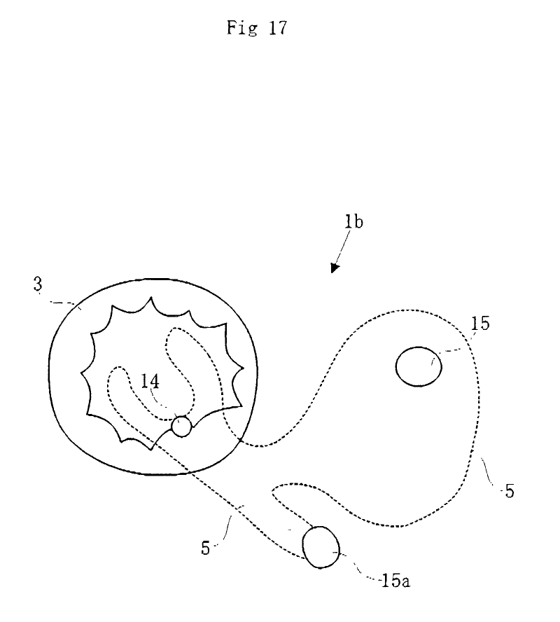

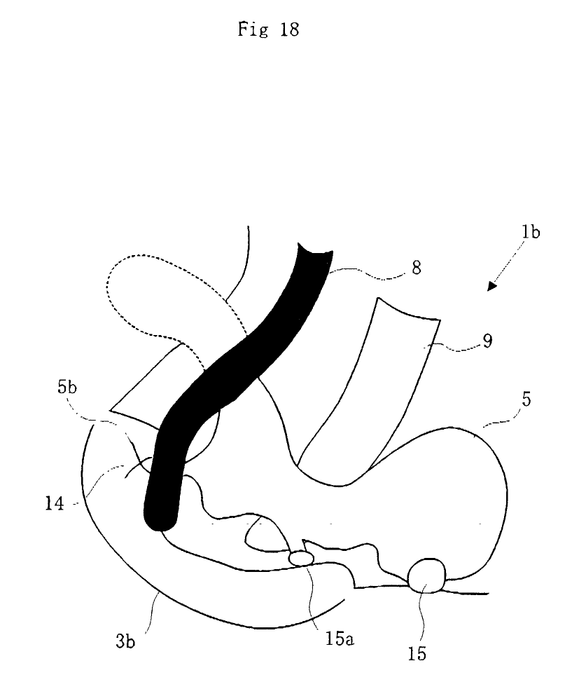

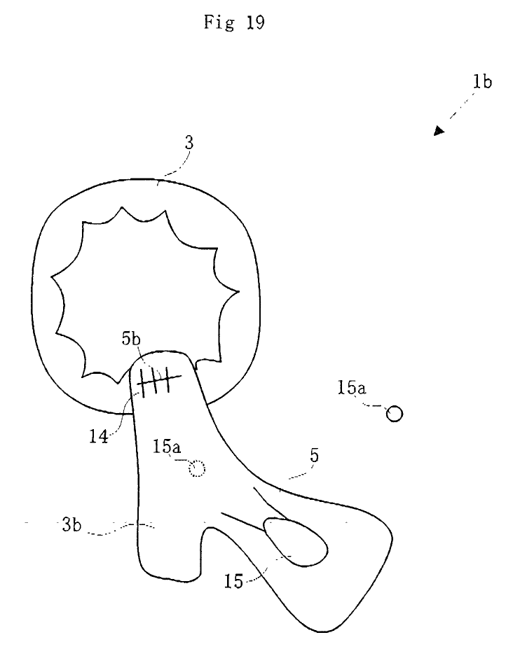

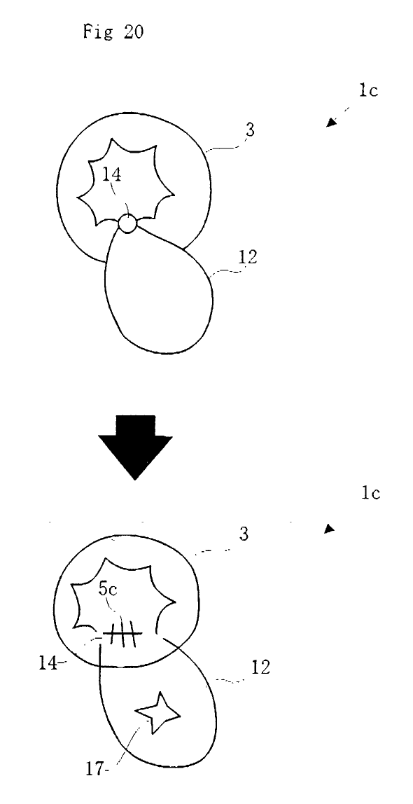

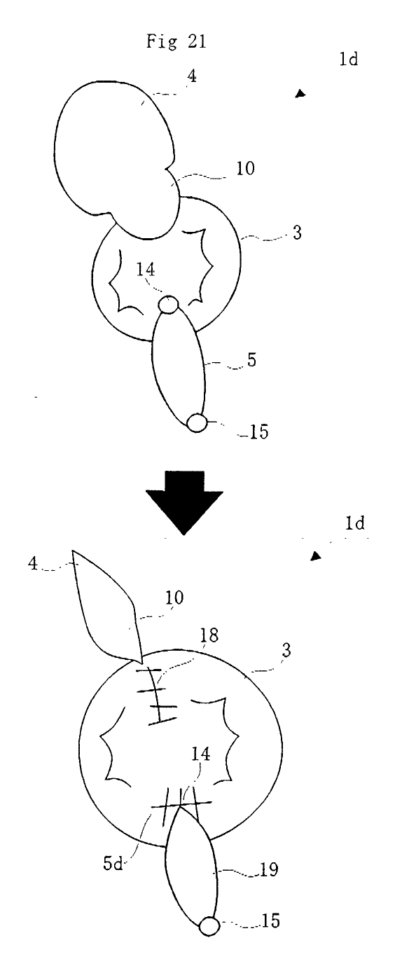

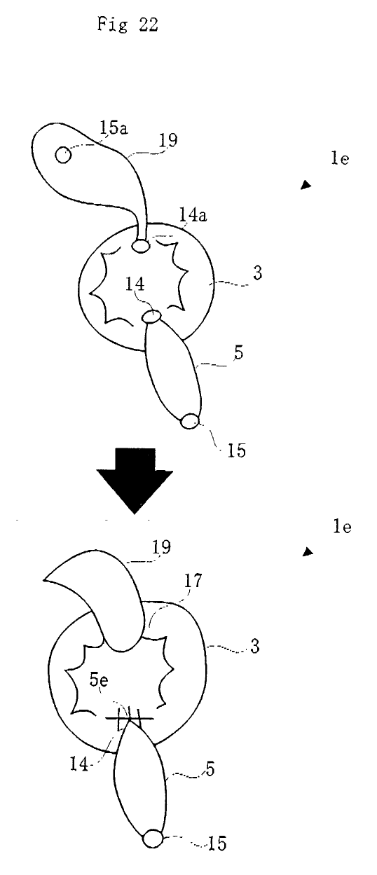

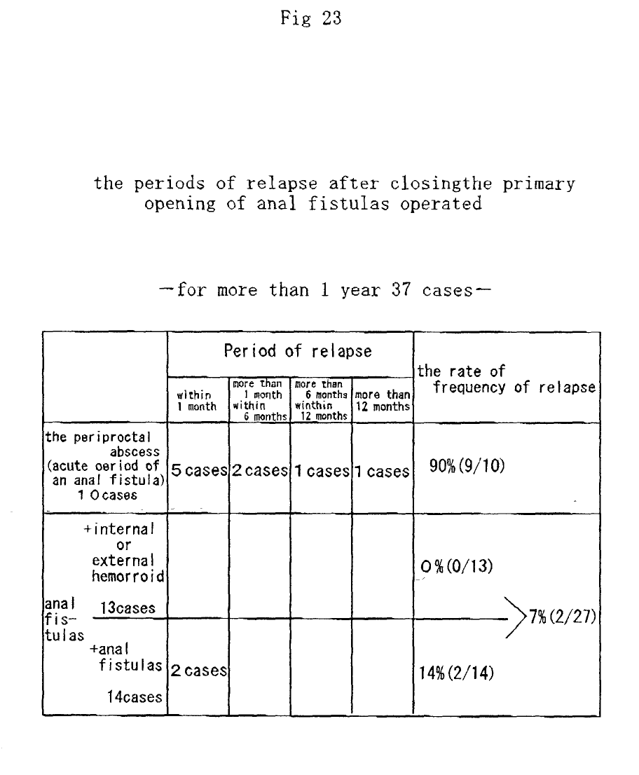

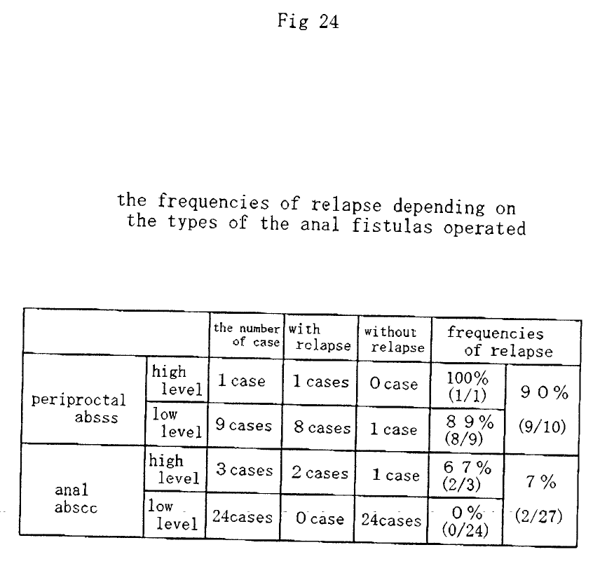

FIG. 16 is an enlarged view showing the case where an anal fistula to he operated has secondary openings developed at two portions, and FIG. 17 is an enlarged bottom view showing the case where the anal fistula to be operated has the secondary openings developed at the two portions. As shown in FIGS. 16 and 17, in the case Of the anal fistula portion 1b where the primary opening 14 is developed at the one portion as well as the secondary openings 15, 15a are developed at the two portions in a surround 3a of an anus 3, an operation method shown in FIGS. 18 and 19 is carried out.

FIG. 18 is an enlarged view showing a state where a primary opening is closed in the case where an anal fistula to be operated has secondary openings developed at two portions and FIG. 19 is an enlarged bottom view showing a state where the primary opening is closed in the case where the anal fistula to be operated has the secondary openings developed at 2 positions.

In the operation method when there are secondary openings 15, 15a at the two portions, first, the primary opening 14 is closed 5b and a drainage hole is formed at the same time by cutting away a lower portion 3b of the anal fistula 5. Through this method of operation, the anal fistula shown in FIGS. 16 and 17 is healed.

FIG. 20 are a bottom view of an anal at an acute period of an anal fistula, which is a periproctal abscess to be operate; and a bottom view showing a state where a primary opening thereof is closed. The periproctal abscess 12 refer to a stage at which bacteria has invaded through an anal fovea 7 getting purulent to form an abscess (a mass of pus). As shown in FIG. 20, the primary opening 14 of the periproctal abscess 2 is closed 5c in order to make the periproctal abscess 12 vanished. Then, a part of the periproctal abscess 12 is incised 17.

By the method of operation where the primary opening S is closed to prevent invasion of bacteria and a part of the periproctal abscess 12 i s incised 17, the invasion of bacteria via the primary opening 14 is prevented, the periproctal abscess 12 can he vanished, and the periproctal abscess 12 can also be vanished without cutting away an internal sphincter 8 and an external sphincter 9.

FIG. 21 are a bottom view of an anus to be operated where an internal hemorrhoid and an external hemorrhoid and an anal fistula are developed, ligation and resection of the internal hemorrhoid and the external hemorrhoid is carried out, and a primary opening of an anal fistula is closed. As shown in FIG. 21, the external hemorrhoid 4 and the internal hemorrhoid 10 are developed in an anus portion 1d and the anal fistula 5 with the primary opening 14 and a secondary opening 15 are also developed therein, and the method is such that these external hemorrhoid 4, internal hemorrhoid 10 and the anal fistula 5 are removed to heal up by means of the operation for the anal fistula according to the present invention.

In this case, first, the internal and external hemorrhoids 4, 10 are ligated and subsequently cut away. Namely, after ligating 18, the intern,al and external hemorrhoids 4, 10 are removed. Then, closed is the primary opening 14 of the anal fistula 19 developed in the anus portion 1d.

FIG. 22 is a bottom view of an anus to be operated where an anal fistula and an anal fistula are developed and a bottom view showing a state where one anal fistula is incisionally released and a primary opening of the other anal fistula is closed. As shown in FIG. 22, shown is an operation method in the case where anal fistulas 5, 19 are developed at two portions in an anal fistula portion 1e. One anal fistula 5 of the anal fistulas developed at the two portions has the primary opening 14 and a secondary opening 15, and the other anal fistula 19 has a primary opening 14a and a secondary opening 15a. The method is such that anal fistulas 5, 19 like this are developed and these anal fistula 5, 19 are removed to heal up by means of the operation for the anal fistula according to the present invention.

In this case, first, the primary opening 14 of one hemorrhoid 5 is closed 5e to be removed as well as the other hemorrhoid 19 is incised and released at the same time, namely the method is performed by incisional release 17.

FIG. 23 is a table showing the periods of relapse after closing the primary openings of the 37 cases of anal fistulas operated. As shown in FIG. 23, referring to the periods of relapse for 10 cases of the periproctal abscess (acute period of an anal fistula), within 1 month 5 cases relapsed, more than 1 month and within 6 months 2 cases relapsed, more than 6 months and within 12 months 1 case relapsed. Therefore, the rate of frequency of relapse is 90%.

In the case where anal fistulas and an internal hemorrhoid and an external hemorrhoid are developed, referring to the periods of relapse for 13 cases operated according to the present method of operation for anal fistulas, there is no cases of relapse. Therefore, the rate of frequency of relapse is 0%.

Further, in the case where an anal fistula and an anal fistula are developed, referring to the periods of relapse for 14 cases operated according to the present method of operation for anal fistulas, there are 2 cases of relapse within 1 month. Therefore, the rate of frequency of relapse is 14%.

Accordingly, the rate of frequency of relapse with the rate of frequency of relapse for the cases operated according to the present method of operation for anal fistulas where an anal fistula and an internal hemorrhoid or an external hemorrhoid are developed and the rate of frequency of relapse for the cases operated according to the present method of operation for anal fistulas where an anal fistula and an anal fistula are developed.

FIG. 24 is a table showing the frequencies of relapse depending on the types of the anal fistulas operated. As shown in FIG. 24, in the 1 case for a periproctal abscess at high level, relapse occurred even after the operation according to the present method of operation for anal fistulas Therefore, the rate of frequency of relapse is 100% in this case. And, in the 9 cases for a periproctal abscess at low level, the 8 cases relapsed and only 1 case did not relapse. Therefore, the rate of frequency of relapse is 89% in this case. Thereby, the average rate of frequency of relapse is 90%.

Furthermore, in the 3 cases for an anal fistula at high level, the 2 cases relapsed and the 1 case did not relapse. Therefore, the rate of frequency of relapse is 67% And, in the 24 cases for am anal fistula at low level, no case relapsed. All the 24 cases did not relapse. Thereby, the average rate of frequency of relapse of the two is 7%.

As means for closing the primary opening or means for cutting away the primary opening and closing the fistulous tract, suturing with thread, applying or injecting a medicine on or into the primary opening, or any suturing equipment is utilized.

### Possibility of Industrial Utilization

Since the present invention comprises the constituents described above and since the method of operation comprises closing a primary opening or cutting away the primary opening, an anal fistula car be healed without cutting away internal and external anal sphincters. Further, probability of relapse of the anal fistula operated according to he present invent on will be extremely small.

1. A method of operating for an anal fistula, comprising the step of closing a primary opening of the purulent anal fistula developed round an anus to vanish away the anal fistula.

2. A method of operating for an anal fistula, comprising the steps of closing a primary opening, and cutting away a lower portion of the anal fistula, in the case that secondary openings are developed at two anal fistula portions developed round an anus.

3. A method of operating for an anal fistula, comprising the steps of closing a primary opening of the purulent anal fistula developed round an anus, and incising a part of a periproctal abscess round the anus.

4. A method of operating for an anal fistula, comprising the steps of ligating and cutting away an internal hemorrhoid and an external hemorrhoid, and closing a primary opening of the anal fistula developed in an anus portion, in the case that the anal fistula having the primary opening and a secondary opening in the anus portion is developed, and the internal hemorrhoid and the external hemorrhoid are developed.

5. A method of operating for an anal fistula, comprising the steps of closing and removing a primary opening of one anal fistula, and closing and removing a primary opening of the other anal fistula, in the case that the anal fistulas are developed at two portions round an anus.

6. A method of operation, comprising the steps of cutting away and then closing a primary opening of an anal fistula which is developed and gets worse round an anus, whereby invasion of bacteria is prevented and a drainage is directly formed from the anal fistula.
